(19) United States
(12) Patent Application Publication
(10) Pub. No.: US 2017/0252433 A1
(43) Pub. Date: Sep. 7, 2017

(54) PSMA-RELATED THERAPIES

(71) Applicant: Memorial Sloan Kettering Cancer Center, New York, NY (US)

(72) Inventors: Jan Grimm, New York, NY (US); Charalambos Kaittanis, New York, NY (US)

(73) Assignee: Memorial Sloan Kettering Cancer Center, New York, NY (US)

(21) Appl. No.: 15/518,010

(22) PCT Filed: Oct. 9, 2015
(86) PCT No.: PCT/US2015/054937

§ 371 Date: Apr. 10, 2017

Related U.S. Application Data
(60) Provisional application No. 62/062,710, filed on Oct. 10, 2014.
(60) Provisional application No. 62/062,714, filed on Oct. 10, 2014.

(51) Int. Cl. A61K 39/395; A61K 31/4166; A61K 31/47; A61K 31/4745; A61K 31/337; A61K 31/5377; A61K 31/662; A61K 31/69; A61K 31/704; A61K 38/05; A61K 31/416; A61K 31/517

(52) U.S. Cl. CPC A61K 39/395; A61K 31/416; A61K 31/4166; A61K 31/47; A61K 31/4745; A61K 31/517; A61K 31/5377; A61K 31/662; A61K 31/69; A61K 31/704; A61K 38/05; A61K 31/337; A61K 45/06

(57) ABSTRACT

The present invention provides methods of treating disease by modulation of PSMA activity. Such modulations can lead to, for example, alterations in cancer tumor metabolism, oxygenation, vascularization, and metastasis. The present invention encompasses the recognition that PSMA, through its role in a complex signaling cascade, can affect cancer progression, angiogenesis, and neovascularization. The present invention provides, among other things, methods of treating cancer, including but not limited to cancer initiation, progression, metastasis, and vascularization by modulation of PSMA activity.

### CROSS REFERENCE TO RELATED APPLICATIONS

This application claims priority to U.S. Provisional Applications 62/062,710 and 62/062,714 both filed Oct. 10, 2014, which are herein incorporated by reference in their entirety.

### BACKGROUND

Prostate cancer is one of the most frequently diagnosed cancers in men, and is the most common cause of cancer-related death after lung cancer. The risk of developing prostate cancer increases dramatically with age, particularly for men over 50. With an aging population and increases in life expectancy that have marked the last thirty years, the incidence rate of prostate cancer in the United States is approaching one in six men.

Early diagnosis and successful treatment of prostate cancer continues to be a major clinical challenge. Apart from new technologies that accurately detect prostatic lesions, understanding significant molecular cascades during prostate carcinogenesis, metastasis, and drug resistance are critical for the development of new therapeutic agents and intervention strategies. A molecular prostate cancer hallmark is the aberrant expression of the transmembrane glycoprotein prostate-specific membrane antigen (PSMA) at the plasma membrane of almost every prostatic neoplasia. PSMA's expression profile on prostate cancers and its enzymatic activity suggest that it might play an important role in prostate cancer and is amenable to pharmacological interventions. The ability to modulate PSMA levels and PSMA's enzymatic activity could be useful in the treatment of cancer and other diseases and conditions mediated by PSMA.

### SUMMARY

The present invention encompasses the recognition that PSMA, through its role in a complex signaling cascade, can affect cancer progression, angiogenesis, and neovascularization. The present invention provides, among other things, methods of treating cancer, including but not limited to cancer initiation, progression, metastasis, and vascularization by modulation of PSMA activity. The present invention also encompasses the recognition that PSMA activity can modulate cytoplasmic calcium levels. Such modulations can lead to alterations in tumor metabolism, oxygenation, vascularization, and metastasis. Thus, according to one aspect of the present invention, PSMA can be utilized as a novel component of therapy.

In some embodiments, the present invention relates to methods of treating or preventing cancer that include administering a therapeutically effective amount of a chemotherapeutic to a patient who is sensitized to the chemotherapeutic in that the patient has received a PSMA inhibitor. In some embodiments, the present invention provides methods of treating or preventing cancer comprising administering to a subject suffering from or susceptible to a refractory cancer a therapeutically effective amount of a PSMA inhibitor. In some embodiments, the present invention provides methods of treating or preventing cancer comprising steps of i) identifying a patient suffering from or susceptible to a cancer characterized by high levels of PSMA; and ii) administering a therapeutically effective amount of a PSMA inhibitor.

In certain embodiments, the present invention provides methods for reducing resistance to a chemotherapeutic in a patient comprising administering a therapeutically effective amount of a PSMA inhibitor concurrent with or prior to administration of the chemotherapeutic. In some embodiments, the present invention provides methods for sensitizing tumor cells to a chemotherapeutic comprising treating the tumor cells with a PSMA inhibitor.

In some embodiments, the present invention provides methods of inhibiting cancer cell migration comprising administering to a patient suffering from or susceptible to cancer a therapeutically effective amount of a PSMA inhibitor.

In certain embodiments, the present invention provides methods of inhibiting neovascularization comprising administering to a patient suffering from or susceptible to cancer a therapeutically effective amount of a PSMA inhibitor. In certain embodiments, the neovascularization is tumor neovascularization.

In certain embodiments, the present invention provides a method of treating cancer in a patient suffering from or susceptible to the cancer, comprising steps of i) determining the amount of PSMA present on a patient's tumor; and ii) administering a suitable chemotherapeutic to the patient; wherein a high level of PSMA indicates the patient should be treated with an elevated level of chemotherapy.

In some embodiments, the present invention provides a method of treating cancer in a patient suffering from or susceptible to cancer, the method comprising steps of administering an elevated dose of achemotherapeutic agent to a patient who: a) is receiving therapy with the chemotherapeutic agent; and b) shows a high level of PSMA.

### Definitions

The term “administration” as used herein refers to the administration of a composition to a subject. Administration may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal, and vitreal.

The term “angiogenesis” as used herein refers to the promotion or development of new capillary blood vessels from pre-existing vessels, resulting in an increased vascularization, often associated with a particular organ or tissue, or with a tumor.

The terms “cancer” and “cancerous”, as used herein, refer to or describe a physiological, histological, or genetic condition in a subject that is characterized by unregulated cell growth or division. In some embodiments, a cancer is a solid tumor. In some embodiments, a cancer is a sarcoma, melanoma, blastoma, or carcinoma. In some embodiments, a cancer is squamous cell cancer (e.g. epithelial squamous cell cancer), lung cancer including small-cell lung cancer, non-small cell lung cancer, adenocarcinoma of the lung and squamous carcinoma of the lung, bone cancer, cancer of the peritoneum, esophageal cancer, eye cancer, skin cancer, gastric or stomach cancer including gastrointestinal cancer, pancreatic cancer, glioblastoma, cervical cancer, ovarian cancer, liver cancer, bladder cancer, gallbladder cancer, hepatoma, laryngeal cancer, oral cancer, brain cancer, breast cancer, colon cancer, rectal cancer, colorectal cancer, endometrial or uterine cancer, salivary gland carcinoma, kidney or renal cancer, neuroendocrine cancer, prostate cancer, vaginal cancer, vulval cancer, testicular cancer, thyroid cancer, urethral cancer, hepatic carcinoma, anal carcinoma, penile carcinoma, as well as head and neck cancer.

The term “chemotherapeutic” as used herein refers to agents having cytostatic and/or cytocidal function and which are useful in the treatment or prevention of cancer and/or neovascularization. In some embodiments, such chemotherapeutics include, but are not limited to, antiproliferative antibodies, topoisomerase I inhibitors; topoisomerase II inhibitors; microtubule active compounds; compounds which induce cell differentiation processes; compounds targeting/decreasing a protein or lipid kinase activity and further anti-angiogenic compounds; compounds which target, decrease, or inhibit the activity of a protein or lipid phosphatase; anti-androgens; proteasome inhibitors; MEK inhibitors such as ARRY142886 from Array BioPharma, AZD6244 from AstraZeneca, PD181461 from Pfizer, and leucovorin. In some embodiments, a chemotherapeutic is selected from DNA intercalators (doxorubicin and its derivatives), mitotic inhibitors (taxol and its derivatives), ERK/MEK inhibitors (e.g., AZD6244 and the alike), dual PI3K/mTOR inhibitors (e.g., BEZ235 and the alike), PI3K inhibitors (e.g., BKM120, GDC-0941, CAL-101, PI-103, XL147, ZSTK474, BYL719, GSK458, PF-04691502, AZD6482, Apitolisib, GSK2636771, Copanlisib), mTOR inhibitors (e.g., Everolimus, AZD8055), EGFR/ErbB2 inhibitors (e.g., lapatinib and the alike), 20S proteasome inhibitors/ROS upregulators (e.g., velcade), AR antagonists (e.g., bicalutamide, galeterone, flutamide, cyproterone acetate, spironolactone), or AR inhibitors (e.g., enzalutamide, anti-androgens, ARN-509, S7040, abiraterone).

The term “antiproliferative antibodies” as used herein includes, but is not limited to, trastuzumab (Herceptin™), Trastuzumab-DM1, cetuximab (Erbitux®), bevacizumab (Avastin™), rituximab (Rituxan®), PRO64553 (anti-CD40), ipilimumab (MDX-101, Yervoy®), panitumumab (Vectibix®), and 2C4 Antibody. By antibodies is meant intact monoclonal antibodies, polyclonal antibodies, multispecific antibodies formed from at least 2 intact antibodies, and antibodies fragments so long as they exhibit the desired biological activity. Such antiproliferative antibodies include antibody-drug conjugates, and may comprises radioactive particles or other chemotherapeutics, for example ado-trastuzumab emtansine (Kadcyla®).

The term “anti-androgen” as used herein relates to any substance which is capable of inhibiting the biological effects of androgenic hormones and includes, but is not limited to, bicalutamide (Casodex™).

The term “topoisomerase I inhibitor” as used herein includes, but is not limited to topotecan, gimatecan, irinotecan, camptothecian and its analogues, 9-nitrocamptothecin, and the macromolecular camptothecin conjugate PNU-166148. Irinotecan can be administered, e.g. in the form as it is marketed, e.g. under the trademark Camptosar™. Topotecan is marketed under the trade name Hycamptin™.

The term “topoisomerase II inhibitor” as used herein includes, but is not limited to the anthracyclines such as doxorubicin (including liposomal formulation, such as Caelyx™), doxorubicin derivatives, daunorubicin, epirubicin, idarubicin, and nemorubicin, the anthraquinones mitoxantrone and losoxantrone, and the podophillotoxines etoposide and teniposide. Etoposide is marketed under the trade name Etopophos™. Teniposide is marketed under the trade name VM 26-Bristol Doxorubicin is marketed under the trade name Acriblastin™ or Adriamycin™. Epirubicin is marketed under the trade name Farmorubicin™. Idarubicin is marketed under the trade name Zavedos™. Mitoxantrone is marketed under the trade name Novantron™.

The term “microtubule active agent” relates to microtubule stabilizing, microtubule destabilizing compounds, and microtublin polymerization inhibitors including, but not limited to taxanes, such as paclitaxel and docetaxel; vinca alkaloids, such as vinblastine or vinblastine sulfate, vincristine or vincristine sulfate, vinflunine, and vinorelbine; discodermolides; cochicine, and epothilones and derivatives thereof. Paclitaxel is marketed under the trade name Taxol™ and Abraxane®. Docetaxel is marketed under the trade name Taxotere™. Vinblastine sulfate is marketed under the trade name Vinblastin R.P™. Vincristine sulfate is marketed under the trade name Farmistin™.

The terms “compounds targeting/decreasing a protein or lipid kinase activity”, or “compounds which target, decrease, or inhibit protein or lipid phosphatase activity”, or “further anti-angiogenic compounds” as used herein includes, but are not limited to, protein tyrosine kinase and/or serine and/or threonine kinase inhibitors or lipid kinase inhibitors, such as a) compounds targeting, decreasing, or inhibiting the activity of the platelet-derived growth factor-receptors (PDGFR), such as compounds which target, decrease, or inhibit the activity of PDGFR, especially compounds which inhibit the PDGF receptor, such as an N-phenyl-2-pyrimidine-amine derivative, such as imatinib, SU101, SU6668 and GFB-111; b) compounds targeting, decreasing, or inhibiting the activity of the fibroblast growth factor-receptors (FGFR); c) compounds targeting, decreasing, or inhibiting the activity of the insulin-like growth factor receptor I (IGF-IR), such as compounds which target, decrease, or inhibit the activity of IGF-IR, especially compounds which inhibit the kinase activity of IGF-I receptor, or antibodies that target the extracellular domain of IGF-I receptor or its growth factors; d) compounds targeting, decreasing, or inhibiting the activity of the Trk receptor tyrosine kinase family, or ephrin B4 inhibitors; e) compounds targeting, decreasing, or inhibiting the activity of the AxI receptor tyrosine kinase family; f) compounds targeting, decreasing, or inhibiting the activity of the Ret receptor tyrosine kinase; g) compounds targeting, decreasing, or inhibiting the activity of the Kit/SCFR receptor tyrosine kinase, such as imatinib; h) compounds targeting, decreasing, or inhibiting the activity of the C-kit receptor tyrosine kinases, which are part of the PDGFR family, such as compounds which target, decrease, or inhibit the activity of the c-Kit receptor tyrosine kinase family, especially compounds which inhibit the c-Kit receptor, such as imatinib; i) compounds targeting, decreasing, or inhibiting the activity of members of the c-Abl family, their gene-fusion products (e.g. BCR-Abl kinase) and mutants, such as compounds which target decrease or inhibit the activity of c-Abl family members and their gene fusion products, such as an N-phenyl-2-pyrimidine-amine derivative, such as imatinib or nilotinib (AMN107); PD180970; AG957; NSC 680410; PD173955 from ParkeDavis; or dasatinib (BMS-354825); j) compounds targeting, decreasing, or inhibiting the activity of members of the protein kinase C (PKC) and Raf family of serine/threonine kinases, members of the MEK, SRC, JAK, FAK, PDK1, PKB/Akt, and Ras/MAPK family members, and/or members of the cyclin-dependent kinase family (CDK) including staurosporine derivatives, such as midostaurin; examples of further compounds include UCN-01, safingol, BAY 43-9006, Bryostatin 1, Perifosine; llmofosine; RO 318220 and RO 320432; GO 6976; lsis 3521; LY333531/LY379196; isochinoline compounds; FTIs; PD184352 or QAN697 (a P13K inhibitor) or AT7519 (CDK inhibitor); k) compounds targeting, decreasing, or inhibiting the activity of protein-tyrosine kinase inhibitors, such as compounds which target, decrease, or inhibit the activity of protein-tyrosine kinase inhibitors include imatinib mesylate (Gleevec™) or tyrphostin such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); 1) compounds targeting, decreasing or inhibiting the activity of the epidermal growth factor family of receptor tyrosine kinases (EGFR₁ ErbB2, ErbB3, ErbB4 as homo- or heterodimers) and their mutants, such as compounds which target, decrease, or inhibit the activity of the epidermal growth factor receptor family are especially compounds, proteins, or antibodies which inhibit members of the EGF receptor tyrosine kinase family, such as EGF receptor, ErbB2, ErbB3, and ErbB4 or bind to EGF or EGF related ligands, CP 358774, ZD 1839, ZM 105180; trastuzumab (Herceptin™), cetuximab (Erbitux™), Iressa, Tarceva, OSI-774, C1-1033, EKB-569, GW-2016, E1.1, E2.4, E2.5, E6.2, E6.4, E2.11, E6.3 or E7.6.3, and 7H-pyrrolo-[2,3-d]pyrimidine derivatives; and m) compounds targeting, decreasing, or inhibiting the activity of the c-Met receptor, such as compounds which target, decrease, or inhibit the activity of c-Met, especially compounds which inhibit the kinase activity of c-Met receptor, or antibodies that target the extracellular domain of c-Met or bind to HGF.

The term “mTOR inhibitors” relates to compounds which inhibit the mammalian target of rapamycin (mTOR) and which possess antiproliferative activity such as sirolimus (Rapamune®), everolimus (Certican™), CCI-779, AZD8055, BEZ235, Temsirolimus, KU-0063794, PP242, Ridaforolimus, INK127, XL765, Torin1, Torin 2, OSI-027, WYE-354, AZD2014, Palomid 529, WAY-600, and ABT578.

The term “proteasome inhibitor” as used herein refers to compounds which target, decrease or inhibit the activity of the proteasome. Compounds which target, decrease, or inhibit the activity of the proteasome include, but are not limited to, Bortezomib (Velcade™) and MLN 341.

The term “in vitro” as used herein refers to events that occur in an artificial environment, e.g., in a test tube or reaction vessel, in cell culture, etc., rather than within an organism (e.g., animal, plant, and/or microbe).

The term “in vivo” as used herein refers to events that occur within an organism (e.g., animal, plant, and/or microbe).

The term “metastasis” as used herein refers to tumor cell entry into and survival in the circulatory system, extravasation, and finally, establishment of distant tumors in secondary organs or tissues.

The term “neovascularization” as used herein refers to the formation of new blood vessels in tissue not normally containing them, especially in tissues where circulation has been impaired by disease or trauma. Non-limiting examples of such disease or trauma include tumors, diabetic retinopathy, arthritis, and psoriasis.

The terms “patient”, “subject”, or “test subject” as used herein refer to any organism to which an inhibitor of PSMA is administered alone or in combination with a chemotherapeutic in accordance with the present invention e.g., for experimental, diagnostic, prophylactic, and/or therapeutic purposes. Typical subjects include animals (e.g., mammals such as mice, rats, rabbits, non-human primates, and humans; insects; worms; etc.). In some embodiments, a subject may be suffering from, and/or susceptible to a disease, disorder, and/or condition (e.g. a cancer, macular degeneration, diabetic retinopathy)

An individual who is “suffering from” a disease, disorder, or condition has been diagnosed with and/or exhibits or has exhibited one or more symptoms or characteristics of the disease, disorder, or condition.

An individual who is “susceptible to” a disease, disorder, or condition is at risk for developing the disease, disorder, or condition. In some embodiments, an individual who is susceptible to a disease, disorder, or condition does not display any symptoms of the disease, disorder, or condition. In some embodiments, an individual who is susceptible to a disease, disorder, or condition has not been diagnosed with the disease, disorder, and/or condition. In some embodiments, an individual who is susceptible to a disease, disorder, or condition is an individual who has been exposed to conditions associated with development of the disease, disorder, or condition. In some embodiments, a risk of developing a disease, disorder, and/or condition is a population-based risk (e.g., family members of individuals suffering from allergy, etc.

The term “therapeutically effective amount” as used herein means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount.

The term “treat,” “treatment,” or “treating” as used herein refers to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition.

The term “tumor” as used herein, refers to an abnormal mass of tissue or collection of cells that results from excessive and abnormal cell division. They may be either benign (not cancerous) or malignant (cancerous).

### BRIEF DESCRIPTION OF THE DRAWINGS

FIG. 1 demonstrates the role that PSMA plays in regulating calcium homeostasis in prostate cancer cells.

FIG. 2 shows the co-localization of PSMA and mGluR1/5 at the plasma membrane of prostate cancer cells.

FIG. 3 shows the enzymatic activity of PSMA can upregulate the phosphorylation of several calcium-dependent signaling effectors.

FIG. 4 shows the importance of PSMA in regulating the phosphorylation of many cellular components involved in oncogenisis.

FIG. 5 demonstrates exemplary mechanisms by which PSMA contributes to the advancement of prostate cancer.

FIG. 6 shows the effects of PSMA activity on angiogenesis and tumor oxygenation.

FIG. 7 demonstrates the increase in cytotoxicity of several chemotherapeutics by the inhibition of PSMA activity.

FIG. 8 shows that PSMA plays a role in resistance to chemotherapeutics that increase the intracellular level of reactive oxygen species.

FIG. 9 demonstrates that tumor growth is reduced and animal survival is increased through the inhibition of PSMA. FIG. 9a shows the percent of tumor free animals treated with vehicle (black line) or 2-PMPA (light gray). FIG. 9b demonstrates the overall survival of animals treated with vehicle (black), having reduced expression of PSMA (LNCaP-KD, light black), or treated with high (light gray) and low (gray) doses of 2-PMPA.

FIG. 10 shows the correlation between PSMA level and response to chemotherapy.

FIG. 11 demonstrates the identification of genes whose expression is upregulated by PSMA expression and activity.

FIG. 12 demonstrates correlation of PSMA expression level with activation of the mTOR pathway.

FIG. 13 shows a model demonstrating the role PSMA plays in regulating various cellular processes that effect tumor growth and progression.

### DETAILED DESCRIPTION OF CERTAIN EMBODIMENTS

PSMA is a transmembrane glutamate carboxypeptidase that is found in prostate cancers and the neovasculature of most solid tumors, but is absent from healthy prostate gland and normal vessels. The expression of PSMA correlates with disease stage and biochemical recurrence and can be used as a biomarker for disease state. In some embodiments, the present invention provides methods of treating or preventing cancer or cancer progression through the analysis of expression of PSMA and/or inhibition of the enzymatic activity of PSMA.

The present invention encompasses the recognition that the expression of PSMA provides resistance to many drugs, but inhibition of PSMA's enzymatic activity sensitizes the cells to these chemotherapeutics. PSMA expression also can render tumors resistant to combinatorial therapy whereas inhibition of PSMA in vivo can result in smaller tumor volume and slower tumor growth. Therefore, one aspect of the present invention provides a method of treating or preventing cancer that includes administering to a subject suffering from or susceptible to a refractory cancer a therapeutically effective amount of a PSMA inhibitor. In some embodiments, the present invention provides methods for reducing resistance to a chemotherapeutic or sensitizing a tumor cell to a chemotherapeutic in a patient through administration of a therapeutically effective amount of a PSMA inhibitor concurrent with or prior to administration of the chemotherapeutic.

The present disclosure details the role of PSMA in modulating growth of tumors and their susceptibility to chemotherapeutics, and how the expression level of PSMA on a patient's tumor can be utilized as a diagnostic to evaluate susceptibility to chemotherapeutics and/or a need for PSMA inhibition. Accordingly, some embodiments of the present invention provide a method of treating or preventing cancer in which patients suffering from or susceptible to a cancer characterized by high levels of PSMA are identified and administered a therapeutically effective amount of a PSMA inhibitor.

Neovascularization is a critical step in a tumor's ability to increase in size. Metastasis is an important hallmark of cancer progression. As detailed in the ensuing Examples, PSMA activity is linked to both of these functions. Therefore, in certain embodiments the present invention provides a method of inhibiting cancer cell migration and/or neovascularization by administering to a patient suffering from or susceptible to cancer a therapeutically effective amount of a PSMA inhibitor.

Inhibition of PSMA's enzymatic activity lowers levels of metastatic effectors, like prostaglandins and VEGF, while switching the cell's primary energy source from oxidative phosphorylation to aerobic glycolysis. Also, inhibition of PSMA upregulates the levels of the archetypic androgen receptor target prostate-specific antigen (PSA). Without wishing to be bound by any particular theory, it is believe that this might be at least partly attributed to PSMA's ability to activate the PI3K-Akt pathway, which negatively regulates the androgen receptor (AR) pathway in prostate cancer. Through its enzymatic activity, PSMA activates downstream signaling involving PI3K and Akt. As a result, the activity and output of the AR pathway, measured in the form of PSA levels, decreases. Alternatively, once PSMA's enzymatic activity is inhibited, the repression by PI3k and AKT over AR decreases, which increases AR signaling, reflected in higher PSA concentration.

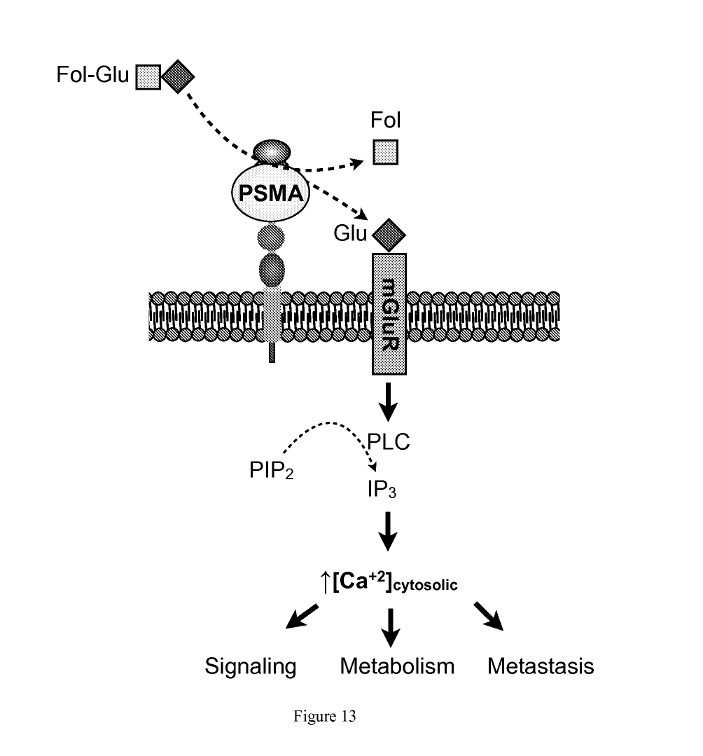

Without wishing to be bound to any particular theories the present disclosure demonstrates that PSMA, through its enzymatic activity and ability to process (poly)glutamated substrates, including NAAG and folates, activates metabotropic Glutamate Receptors Group I, which initiate a downstream signaling cascade that increases cytosolic calcium levels. The released calcium further activates various signaling effectors, alters metabolism and primes the tumor and its environment for metastasis. See FIG. 13.

Any PSMA inhibitor can be used in accordance with the present invention. PSMA inhibitors are known in the art, for example (RS)-2-PMPA, (R)-2-PMPA, (S)-2-PMPA, (RS)-GPI5232, (S)-GPI5232, RS)-2-MMPA, (R)-2-MMPA, (S)-2-MMPA, PBDA, (R,R)/(S,S)-PBDA, (S,S)/(R,R)-PBDA, meso-PBDA, (S)-Glu-C(O)-(S)-Glu, (R)-Glu-C(O)-(R)-Glu, (R)-Glu-C(O)-(S)-Glu, [¹¹C]DCMC, [¹²⁵I]DCIT, VA-033, ZJ43, ZJ11, ZJ17, ZJ38 (Zhou J, Neale J H, Pomper M G, Kozikowski A P. Nat Rev Drug Discov. 2005 December; 4(12):1015-26.); CTT54 (Kasten B. B. et al, Bioorg Med Chem Lett. 2013 Jan. 15; 23(2):565-8); TG97 and DBCO-PEG₄-AH₂-TG97 (Martin S. E. et al, Bioconjug Chem. 2014 Sep. 15); DBCO-PEG(4)-CTT-54, DBCO-PEG(4)-CTT-54.2 (Nedrow-Byers et al, Prostate, 2013); beta-NAAG (Yourick D. L. et al, Brain Res. 2003 Nov. 21; 991(1-2):56-64); pemetrexed, methotrexate (Fulton M. D. and Berkman C. E., Pacific Northwest Research Symposium, 2011, retrieved from chemistry.oregonstate.edu/organic/symposium/archive/2011/abstracts2011/abs_fulton.pdf); pseudoirreversible inhibitor peptidomimetics (Liu T. et al, Biochemistry. 2008 Dec. 2; 47(48):12658-60); and steroid-derived phosphoramidate inhibitors (Wu L. Y. et al, Biochemistry. 2008 Dec. 2; 47(48):12658-60). In some embodiments, a PSMA inhibitor is a PSMA inhibitor as described in any of the references cited in this paragraph, the entire contents of each of which are hereby incorporated by reference herein. In certain embodiments, a PSMA inhibitor is 2-PMPA or an analog thereof.

In some embodiments, a PSMA inhibitor is an alphabody (i.e., a polypeptide that may be tuned to have high affinity toward a target of interest). The production and selection of alphabodies is known in the art, an example of which is described in WO/2012093172, the entire contents of which are hereby incorporated by reference herein.

In some embodiments, a PSMA inhibitor is a DARPin (i.e., Designed Ankyrin, Repeat Protein with specific, high-affinity target binding). DARPins are known in the art and described for example by Binz et al. (Nat Biotechnol. 2004 May; 22(5):575-82) and Stumpp and Amstutz (Curr Opin Drug Discov Devel. 2007 March; 10(2):153-9), the entire contents of each of which are hereby incorporated by reference herein.

In some embodiments, a PSMA inhibitor can act as a competitive inhibitor. In other embodiments the inhibitor may be a non-competitive inhibitor or an allosteric inhibitor. In some embodiments a PSMA inhibitor or portion thereof may be conjugated to a useful detectable agent such as but not limited to a fluorescent group or a radioisotope.

### Treatment of Cancer

PSMA has an enzymatic activity as a glutamate carboxypeptidase. The enzymatic activity is involved in the hydrolytic cleavage and liberation of glutamate from substrates such as glutamyl derivatives of folic acid and N-acetylaspartylglutamate (NAAG). Glutamate liberated by the enzymatic activity of PSMA can activate metabotropic glutamate receptors (mGluRs) some which have been found to co-localize with PSMA (mGluR1 and mGluR5). One component of activation of these receptors is the increase in cytosolic calcium concentrations through inositol triphosphate formation. Some cancers, such as melanoma, overexpress mGluR2 and mGluR3, and PSMA may play a role through activation of these receptors.

Increases in intracellular calcium can lead to activation of numerous cellular kinases which broadly effect downstream signaling. These kinases can include but are not limited to the master kinase Calcium/Calmodulin dependent kinase kinase II (CAMKK2) and mTORC2. The ensuing Examples suggest that the enzymatic activity of PSMA can activate CAMKK2 which leads to activation of downstream kinases including but not limited to PI3K, AKT, Src, and p27. The present invention also encompasses the recognition that PSMA regulates the activation of other kinases such as STAT3, STAT5, and WNK1. As these kinases have significant impact on cellular homeostasis, their activation can lead to the regulation of multiple cellular processes that are involved in cell cycle regulation and signal transduction among other pathways critical for tumorigenesis and cancer progression. Indeed, studies disclosed herein show that the activity of PSMA is related to development and advancement of prostate cancer, suggesting that early therapeutic interventions that effectively inhibit PSMA might have great clinical potential and increase survival. In some embodiments, provided methods include the co-administering inhibitors of one or more of these kinases in combination with a PSMA inhibitor. Such inhibitors are known in the art and/or described herein. In some embodiments, inhibitor of STAT3 or STAT5 is selected from WHI-P154, WP1066, Stattic, S3I-201, HO-3867, or nifuroxazide. In certain embodiments, an inhibitor of WNK1 is selected from PP1 or PP2 (see Yagi et al., Biochemistry. 2009 Nov. 3; 48(43):10255-66), the entire content of which are hereby incorporated by reference)

Though current standard of care chemotherapies can be successful, they can also lead to tumor resistance. Mechanisms of chemotherapeutic resistance include but are not limited to matters concerning access of the drug to the tumor, infusion rate and route of delivery as well as mechanisms including drug metabolism and efflux or excretion. The alterations in cellular homeostasis and signaling affected by the activity of PSMA can also affect the sensitivity of a tumor cell to chemotherapeutics. Findings disclosed herein demonstrate the increased cytotoxicity of certain chemotherapeutics when used in combination with inhibitors of PSMA activity.

Given these findings, certain embodiments of the present invention relate to a method of treating or preventing cancer by administering a therapeutically effective amount of a chemotherapeutic to a patient who is sensitized to the chemotherapeutic in that the patient has received a PSMA inhibitor. In some embodiments, the method comprises the step of administering to the patient a therapeutically effective amount of a PSMA inhibitor prior to administration of the chemotherapeutic. In some embodiments, the method comprises the step of administering to the patient a therapeutically effective amount of a PSMA inhibitor concurrent with administration of the chemotherapeutic.

The present invention also provides a method of treating or preventing cancer comprising administering to a subject suffering from or susceptible to a refractory cancer a therapeutically effective amount of a PSMA inhibitor. In some embodiments, the cancer is castration-resistant prostate cancer. In some embodiments, the cancer is refractory to treatment with an androgen receptor inhibitor or hormone deprivation. In some embodiments, the cancer is refractory to a chemotherapeutic agent as defined herein.

In some embodiments, the present invention provides a method for reducing resistance to a chemotherapeutic in a patient comprising administering a therapeutically effective amount of a PSMA inhibitor concurrent with or prior to administration of the chemotherapeutic.

As PSMA expression is a hallmark of prostatic neoplasia and other solid tumors, embodiments of the present invention provide for methods of identifying a patient suffering from or susceptible to a cancer as characterized by a high level of PSMA. The level of PSMA can be determined by numerous tests including but not limited to histology, biopsy, serology, and medical imaging. In some embodiments, a level of PSMA can be determined using radiolabeled tracers, for example antibodies comprising such tracers. In certain embodiments, a level of PSMA can be determined using dye-labeled tracers. In some embodiments, the level of PSMA can be determined by binding of PSMA with radiolabeled or fluorescent tracers such as but not limited to antibodies or small molecules.

In additional embodiments imaging can be achieved through the use of nanoparticles. In some embodiments, such nanoparticles are comprised of a metal, a metal-like material, or a non-metal. In some embodiments, a nanoparticle core may optionally comprise one or more coating layers, surface-associated entities and/or one dopant entities. In some embodiments, nanoparticles may have one or more surface-associated entities such as stabilizing entities, targeting entities, etc. In some embodiments, such surface-associated entities are or are comprised in a layer. In some embodiments, such entities are associated with or attached to a core. In some embodiments, such entities are associated with or attached to a layer. In some embodiments, nanoparticles are bound to medical isotopes layered with a targeting moiety or a dopant. In some embodiments, such nanoparticles comprise a PSMA inhibitor or a portion thereof. In some embodiments, such targeting moieties are antibodies or small molecules. In some embodiments, dopants are fluorochromes (e.g., near infrared (e.g., metal-enhanced) fluorescence agents, 2-photon fluorescence agents, etc. such as Alexa 647, Alexa 488 and the like), laser pumping materials (e.g., consisting of, but not limited to, materials from the group of the rare-earth metal- and/or transition metal-based compounds), luminescent compounds consisting of, but not limited to rare-earth metals and/or transition metals photoacoustic-active dyes, SE(R)RS-active agents, upconverting materials (e.g. consisting of materials from the group of the rare-earth metals and/or transition metals), “slow light”-inducing materials (e.g., praseodymium-based compounds), ultrasound (US) agents, and any combination thereof (see U.S. Pat. Nos. 5,306,403, 6,002,471, and 6,174,677, the entire contents of each of which are hereby incorporated by reference herein). In some embodiments, a level of PSMA is determined from a tissue homogenate. In some embodiments, a level of PSMA is determined from a plasma membrane assay.

PSMA levels may also be measured using a glutamic acid assay as described in the ensuing Examples. In some embodiments, the assay comprises modification of the commercial Amplex Red Glutamic Acid assay where folic acid (pteroyl-L-glutamic acid) is amenable to cleavage by PSMA, providing glutamate as the substrate of the Amplex Red Glutamic Acid assay. Expressed prostatic secretion is incubated with folic acid and Amplex Red Glutamic Acid reagents. PSMA-containing samples then show a positive fluorescence, quantifiable with a fluorescence reader. In some embodiments, the comprises glutamate conjugated to luciferin via an amide bond, which is amenable to cleavage by PSMA. Expressed prostatic secretion is incubated with the glutamate agent, plus ATP and relevant cofactors. PSMA-containing samples then show a positive luminescence, quantifiable with a luminometer.

In some embodiments, it is useful to assay a patient's PSMA levels in order to ascertain the type of cancer and/or candidate treatment regimens. In some embodiments, the present invention provides a method of treating or preventing cancer comprising identifying a patient suffering from or susceptible to a cancer characterized by high levels of PSMA, and administering a therapeutically effective amount of a PSMA inhibitor. In some embodiments, the method further comprises the step of administering a therapeutically effective amount of a chemotherapeutic concurrent with or subsequent to administration of a PSMA inhibitor.

As used herein, the term “high level of PSMA” refers to instances i) when the concentration of PSMA in the patient's test tissue sample is higher than the concentration of PSMA from the patient's healthy tissue sample, or ii) when the concentration of PSMA in the patient's test tissue sample is higher than the normal concentration of PSMA in the patient population. In some embodiments, a healthy tissue sample is healthy prostate tissue or tissue from a benign prostatic hyperplasia. In some embodiments, a high level of PSMA is where a concentration of PSMA in the patient's test tissue sample is at least 2-fold, at least 3-fold, at least 4-fold, at least 5-fold, at least 6-fold, at least 7-fold, at least 8-fold, at least 9-fold, or at least 10-fold higher than the concentration of PSMA in the patient's healthy tissue or the normal concentration of PSMA in the patient population.

In some embodiments, a high level of PSMA is indicated when a patient has a PSMA level above about 1-5 pg/mL. In some embodiments, a high level of PSMA is indicated when a patient has a PSMA level above about 5 pg/mL, about 10 pg/mL, about 20 pg/mL, about 30 pg/mL, about 50 pg/mL, about 75 pg/mL, about 100 pg/mL, about 150 pg/mL, about 200 pg/mL, about 250 pg/mL, about 500 pg/mL, or about 1000 pg/mL. In some embodiments, a high level of PSMA is indicated when a patient has a PSMA level above about 125 ng/mL, about 150 ng/mL, about 175 ng/mL, about 200 ng/mL, about 225 ng/mL, about 250 ng/mL, about 275 ng/mL, about 300 ng/mL, about 325 ng/mL, about 350 ng/mL, about 375 ng/mL, about 400 ng/mL, about 450 ng/mL, or about 500 ng/mL.

In some embodiments, a high level of PSMA is indicated when a sample of expressed prostatic secretion incubated with folic-acid—Amplex Red Glutamic Acid reagents (e.g., as described in the ensuing Examples) shows a fluorescence or luminescence radiance of greater than 50, normalized to volume. In some embodiments, a high level of PSMA is indicated when a sample of expressed prostatic secretion incubated with folic acid—Amplex Red Glutamic Acid reagents or shows a luminescence radiance of greater than 70, 80, 90, 100, 125, 150, or 200, normalized to volume.

In some embodiments, a high level of PSMA is indicated when a sample of expressed prostatic secretion incubated with an activatable agent (e.g., as described in the ensuing Examples) shows a luminescence radiance of greater than 50, normalized to volume. In some embodiments, a high level of PSMA is indicated when a sample of expressed prostatic secretion incubated with an activatable agent shows a luminescence radiance of greater than 70, 80, 90, 100, 125, 150, or 200, normalized to volume.

In addition to patient treatment, methods of the present invention may be used in vitro as well. In some embodiments, the present invention provides a method for sensitizing tumor cells to a chemotherapeutic comprising treating the tumor cells with a PSMA inhibitor.

In provided methods of the invention, a chemotherapeutic is as defined herein. In some embodiments, a chemotherapeutic is selected from topoisomerase I inhibitors, topoisomerase II inhibitors, microtubule active compounds, compounds which induce cell differentiation processes, compounds targeting/decreasing a protein or lipid kinase activity and further anti-angiogenic compounds, compounds which target, decrease, or inhibit the activity of a protein or lipid phosphatase, anti-androgens, proteasome inhibitors, or MEK inhibitors. In some embodiments, a chemotherapeutic is selected from doxorubicin, taxol, AZD6244, BEZ235, lapatinib, velcade, and enzalutamide.

In some embodiments, an effective concentration of a PSMA inhibitor can range from 1-100 nM, 1-500 nM, 1-1000 nM, 1-100 uM, 1-500 uM, 1-1000 uM, 1-5 mM, 2-6 mM, 3-7 mM, 4-8 mM, 5-9 mM, wherein the concentration of the PSMA inhibitor alone is not cytotoxic. In some embodiments, an effective concentration of a PSMA inhibitor can range from 1-1000 mg/m², 1-10 mg/m², 11-50 mg/m², 51-100 mg/m², 101-500 mg/m², or 501-1000 mg/m². In some embodiments, the PSMA inhibitor is used at a concentration that alone slows but does not reverse tumor growth. In some embodiments, the therapeutically effective amount of PSMA inhibitor is an amount effective to inhibit or decrease metastatic spread of cancer.

In the provided methods disclosed above and herein, a cancer can be any cancer as defined herein. In some embodiments, the cancer is of the prostate. In some embodiments, a cancer is of the breast, lung, or colon. In some embodiments, the cancer comprises a solid tumor. In some embodiments, the solid tumor is other than a prostate or sarcoma tumor.

In some embodiments, the cancer to be treated is resistant to treatment with chemotherapeutics, androgen receptor inhibitors, or forms of hormone deprivation.

### Treatment of Neovascularization and Metastasis

Neovascularzation is an important factor in the progression and pathogenesis of several disorders including but not limited to rheumatoid arthritis, diabetic retinopathy, macular degeneration, and tumor growth. In cancer, the formation of new blood vessels allows the tumor cells to divide and eventually leave the original tumor to form new foci elsewhere in the body or metastasize.

The modulation of signaling within cells by PSMA activity has previously been implicated in the process of neovascularization. Specifically, PSMA has been demonstrated to regulate integrin signaling and cytoskeletal dynamics through the modulation of p21 activated kinases (PAK) and focal adhesion kinase (FAK) (Conway et al. 2006). Additionally, examples in the present disclosure demonstrate an increase in VEGF-A concurrent with PSMA expression, and results show that inhibition of PSMA caused lower tumor vascularization and lower tumor oxygenation. These results further demonstrate the importance of PSMA in the ability of tumors to grow and metastasize.

Therefore, certain embodiments of the invention provide a method of inhibiting cancer cell migration, which includes administering to a patient suffering from or susceptible to cancer a therapeutically effective amount of a PSMA inhibitor.

In some embodiments, the present invention provides a method of inhibiting neovascularization including administering to a patient suffering from a disease whose pathogenesis includes neovascularization. Further embodiments provide a method of inhibiting neovascularization including administering to a patient suffering from or susceptible to cancer a therapeutically effective amount of a PSMA inhibitor. Additional embodiments provide a method for inhibiting neovascularization wherein the tumor is, by way of non-limiting example carcinoma, lymphoma, blastoma, and sarcoma. Further embodiments provide for inhibiting neovascularization wherein the tumor is a solid tumor of tissue including but not limited to breast, lung or colon.

It has been found that some anti-angiogenesis treatments have been largely ineffective against cancer as monotherapies, but offer improved outcomes when co-administered in combination with conventional chemotherapy as compared to the conventional chemotherapy alone. This paradoxical effect can be explained by a normalization of the tumor vasculature by the anti-angiogenesis treatment, wherein the vasculature changes from “abnormal” to a more “normal” phenotype. The “abnormal” phenotype in tumors is often characterized by hypoxia and nutrient-deprivation, which can also promote resistance to treatment. Normalization of these vessels can lead to improved delivery and efficacy of exogenously administered therapeutics. Without wishing to be bound by any particular theory, it is believed that the PSMA located on tumor neovasculature can facilitate the abnormal vasculature phenotype by promoting vessel hyperpermeability. Therefore, in some embodiments, the present invention provides a method of normalizing tumor vasculature by the administration of a PSMA inhibitor. In some embodiments, co-administration of a PSMA inhibitor and a chemotherapeutic results in improved treatment of cancer via tumor vasculature normalization.

In some embodiments, provided methods include treating patients suffering from a disease wherein the PSMA inhibitor is used at a concentration that alone slows but does not reverse tumor growth. In some embodiments, the therapeutically effective amount of PSMA inhibitor is an amount effective to inhibit or decrease metastatic spread of cancer.

Additional embodiments provide a method of treating cancer in a patient suffering from or susceptible to the cancer which includes the steps of determining the amount of PSMA present on a patient's tumor; and administering a suitable chemotherapeutic to the patient; wherein a high level of PSMA indicates the patient should be treated with an elevated level of chemotherapy. Additional embodiments provide a method of treating cancer in a patient suffering from or susceptible to the cancer which includes the steps of determining the amount of PSMA present on a patient's tumor; and administering a suitable chemotherapeutic to the patient; wherein a PSMA level above about 1-5 pg/mL indicates the patient should be treated with an elevated level of chemotherapy. In some embodiments, the present invention provides a method of treating cancer in a patient suffering from or susceptible to cancer, the method comprising a step of administering an elevated dose of a chemotherapeutic agent to a patient who: a) is receiving therapy with a chemotherapeutic agent; and b) shows a level of PSMA above about 1-5 pg/mL.

In some embodiments, a PSMA level above about 1-5 pg/mL, about 5 pg/mL, about 10 pg/mL, about 20 pg/mL, about 30 pg/mL, about 50 pg/mL, about 75 pg/mL, about 100 pg/mL, about 150 pg/mL, about 200 pg/mL, about 250 pg/mL, about 500 pg/mL, or about 1000 pg/mL indicates the patient should be treated with an elevated level of chemotherapy.

In some embodiments, a PSMA level above about 125 ng/mL, about 150 ng/mL, about 175 ng/mL, about 200 ng/mL, about 225 ng/mL, about 250 ng/mL, about 275 ng/mL, about 300 ng/mL, about 325 ng/mL, about 350 ng/mL, about 375 ng/mL, about 400 ng/mL, about 450 ng/mL, or about 500 ng/mL indicates the patient should be treated with an elevated level of chemotherapy.

In some embodiments, a patient should be treated with an elevated level of chemotherapy when a high level of PSMA is indicated in a folic acid—Amplex Red Glutamic Acid assay as described above and herein.

In some embodiments, a patient should be treated with an elevated level of chemotherapy when a high level of PSMA is indicated in an activatable agent assay as described above and herein.

In some embodiments, an elevated level of chemotherapy comprises increasing the concentration of one or more chemotherapeutics the patient is administered.

In certain embodiments, an elevated level of chemotherapy is a dose that is greater than a previously administered dose, a recommended dose, or an approved dose. In some embodiments, an elevated level of chemotherapy represents an increase of about 10-200% of a previously administered dose, a recommended dose, or an approved dose. In some embodiments, an elevated level of chemotherapy represents an increase of about 10-100% of a previously administered dose, a recommended dose, or an approved dose. In some embodiments, an elevated level of chemotherapy represents an increase of about 20-100% of a previously administered dose, a recommended dose, or an approved dose. In some embodiments, an elevated level of chemotherapy represents an increase of about 30-100% of a previously administered dose, a recommended dose, or an approved dose. In some embodiments, an elevated level of chemotherapy represents an increase of about 40-100% of a previously administered dose, a recommended dose, or an approved dose. In some embodiments, an elevated level of chemotherapy represents an increase of about 50-100% of a previously administered dose, a recommended dose, or an approved dose. In some embodiments, an elevated level of chemotherapy represents an increase of about 60-100% of a previously administered dose, a recommended dose, or an approved dose. In some embodiments, an elevated level of chemotherapy represents an increase of about 75-100% of a previously administered dose, a recommended dose, or an approved dose. In some embodiments, an elevated level of chemotherapy represents an increase of about 100-200% of a previously administered dose, a recommended dose, or an approved dose.

In some embodiments, an elevated level of chemotherapy is about 1-10 times a previously administered dose, a recommended dose, or an approved dose. In some embodiments, an elevated level of chemotherapy is about 2-10 times a previously administered dose, a recommended dose, or an approved dose. In some embodiments, an elevated level of chemotherapy is about 2-5 times a previously administered dose, a recommended dose, or an approved dose. In some embodiments, an elevated level of chemotherapy is about 1-5 times a previously administered dose, a recommended dose, or an approved dose.

In some embodiments, an elevated level of chemotherapy comprises administering one or more additional chemotherapeutics to the patient. In some embodiments, an additional chemotherapeutic is selected from the group consisting of mitoxantrone, prednisone, docetaxel, dexamethasone, estremustine, warfarin, cabazitaxel, estramustine etoposide, enzalutamide, and BEZ235. In some embodiments, an elevated level of chemotherapy comprises administering a combination of chemotherapeutics. In some embodiments, a combination of chemotherapeutics comprises mitoxantrone and prednisone, docetaxel and prednisone, docetaxel and dexamethasone, estremustine and warfarin, docetaxel and cabazitaxel, estramustine and etoposide, or enzalutamide and BEZ235.

In some embodiments, a previously administered dose is a dose previously administered to a patient prior to determining the amount of PSMA present on the patient's tumor. In some embodiments, a recommended dose is a dose recommended or prescribed by a physician or other medical professional. In certain embodiments, an approved dose is a dose approved by the United States Food and Drug Administration for the chemotherapeutic.

### EXEMPLIFICATION

### Example 1: Materials and Methods

### Cell Culture

All cell lines were obtained from ATCC (Manassas, Va.), and were grown according to the supplier's guidelines. LNCaP and PC3 cells were grown in 10%-fetal-bovine-serum-containing RPMI 1640 medium, which was supplemented with HEPES buffer (1%), penicillin/streptomycin (1%) and sodium pyruvate (1%). The transduced PC3 cells that expressed PSMA were grown in 10%-fetal-bovine-serum-containing F12K medium, which was supplemented with penicillin/streptomycin (1%) and puromycin (6 μg/mL). The transduced LNCaP cells, where PSMA was knocked down, were grown in 10%-fetal-bovine-serum-containing RPMI 1640 medium, which was supplemented with HEPES buffer (1%), penicillin/streptomycin (1%), sodium pyruvate (1%), and puromycin (3 μg/mL).

hPMSA

To transduce PSMA into PC3 cells, the SFG backbone plasmid containing the human PSMA gene under ampicillin selection was transfected into cells. Successfully transduced cells were selected based on resistance to puromycin. To knock down PSMA from LNCaP cell, the shRNA close RLGH-DU53991 was used (Transomic, Huntsville, Ala.).

### Cytoplasmic Calcium Quantification

Cells were seeded at a density of 10,000 per well on a 96-well plate and grown overnight. Loading of the calcium-sensing dye Fluo4 (Life Technologies, Calrsbad, Calif.) was performed according to the manufacturer's instructions. Where indicated the cells were treated with thapsigargin (1 μM, Tocris, Minneapolis, Minn.), L-Quisqualic acid (110 μM, Tocris), 3,5-DHPG (0.67 mM, Tocris), L-AP3 (125 μM, Tocris), U73122 (50 μM, Tocris), and 2-PMPA (100 μM, Tocris) immediately before measuring calcium levels, with a SpectraMax M5 plate reader (Molecular Devices, Sunnyvale, Calif.).

### Kinome Profiling

CAMKK2, AMPKα and AKT antibodies were purchased from Cell Signaling (Danvers, Mass.). Cells were grown to confluence and treated with 2-PMPA (5 μM, Tocris) for 48 h. The phosphorylation status of proteins of interest was determined with an isoelectric-focusing instrument (NanoPro1000, Protein Simple, Santa Clara, Calif.), where the samples were prepared and analyzed according to the supplier's guidelines. Determination of site-specific phosphorylation of key proteins was performed with a human phosphor-kinase array (ARY003, R&D Systems, Minneapolis, Minn.), according to the product's instructions.

### Signal Output Evaluation

Gene expression array was performed after TRIzol-based RNA extraction on an U133A 2.0 gene array (Affymetrix, Santa Clara, Calif.). The output of the androgen receptor (AR) pathway was assessed by measuring the levels of prostate specific antigen (PSA). LNCaP cells that have a functional AR pathway were obtained from ATCC (Manassas, Va.) and grown to confluence. Then they were treated for 48 h with 2-PMPA (5 μM) for 48 h, followed by screening of the culture medium for secreted PSA with the DELFIA PSA assay (Perkin Elmer, Waltham, Mass.). The levels of nitric oxide attributed to nitric oxide synthase's activity and the concentration of total secreted prostaglandins were quantified with the corresponding kits purchased from Cayman Chemicals (Ann Arbor, Mich.). Alterations in energy production due to signaling changes were determined through quantification of the intracellular NAD+ and ATP lelels, using the NAD assay from Cayman Chemicals (Ann Arbor, Mich.) and the StayBrite ATP kit from Biovision (Milpitas, Calif.).

Immunostaining for PSMA and mGluR1/5.

LNCaP cells were seeded on 4-well chamber slides at a density of 1,000 cells per well. After overnight growth, the cells were fixed with 4% paraformaldehyde, followed by consecutive staining with the mouse J591 antibody to detect PSMA's extracellular motif, and a rabbit polyclonal antibody for type I mGluR (mGluR1/5 antibody, NB300-126, Novus Biologicals, Littleton, Colo.). The nucleus was stained with Hoechst 33342 (Life Technologies, Carlsbad, Calif.). The slides were imaged with a Leica upright confocal SP5 microscope.

### Imaging of In Vivo Metabolic Alterations

Male, athymic, nude mice (Harlan Laboratories, Indianapolis, Ind.) were implanted with LNCaP xenografts (3 million cells in 100 μL Matrigel). Immediately post xengraft implantation, the mice were treated daily iv with 2-PMPA (0.4 mM, 100 μL retro-orbital injection). Tumor vascularization and oxygenation was assessed 15 days after treatment commencements, using the Vevo LAZR small-animal photoacoustic imaging platform (Visualsonics, Toronto, ON, Canada).

### In Vitro Chemotherapy

Cells were seeded in a 96-well format (2,500 cells per well), and grown for 48 h. They were then treated with the following drugs at a final concentration of 200 nM: Doxorubicin, Taxol, AZD6244, BEZ235, Lapatinib, and Velcade (all purchased from Selleck Chemicals, Houston, Tex.). For combination therapy, cells were treated with 2-PMPA (5 μM final concentration). After overnight incubation in the presence of the drugs, cell viability was determined with the Alamar blue method according to the supplier's guidelines (Life Technologies, Carlsbad, Calif.)

### In Vivo Chemotherapy

Male, athymic, nude mice (Harlan Laboratories, Indianapolis, Ind.) were implanted with LNCaP xenografts (3 million cells in 100 μL Matrigel) on each flank. Immediately post xengraft implantation, the mice were treated daily iv with 2-PMPA (0.4 mM, 100 μL retro-orbital injection). Tumor volume was assessed by measuring the tumor with microcalipers. Combination therapy was performed with male athymic, nude mice with LNCaP xenografts, which were treated daily post tumor detection. Chemotherapy was administered by iv (100 μL retro-orbital injection) with animals receiving either enzalutamide (0.15 mM), 2-PMPA (3 mM) or both compounds (0.15 mM enzalutamide and 3 mM 2-PMPA). Male athymic, nude mice with PC3-PSMA xenografts (3 million cells in 100 μL Matrigel) were treated with either vehicle (DMSO) or a combination of AZD8055 and XL184 (AZD8055 0.4 mM and XL184 8 μM 100 μL retro-orbital injection).

### Analysis of Human Prostate Cancer Samples

Biochemical recurrence metastasis data were obtained through the cBIO portal (www.cbioportal.org), and the retrieved data were plotted on the data presentation software Prism. Prostate cancer biopsies from patients undergoing prostatectomy were obtained from MSKCC according to institutional guidelines. Samples were processed and placed on glass-slide tissue microarray, and were then stained with the anti-PSMA antibody (DACO), anti-PTEN antibody (Cell Signaling Technology) or anti-4EBP1 (Cell Signaling Technology), using standard immunohistochemistry protocols. Imaging and scoring was performed by an independent pathologist unaffiliated with the study. The pathology results were then processed on MatLab, through principal component analysis for statistical evaluation and pattern identification. PSMA PET imaging was performed at TUM, using a 68Ga-PSMA-specific agent, in prostate cancer patients prior to prostatectomy, and in accordance to institutional procedures. Biopsies from the primary tumor were processed and samples were deposited on glass slides, which were stained with the anti-PSMA antibody (DAKO) and anti-pAKT (Cell Signaling Technology), following standard immunofluorescence microscopy workflow.

Gene Set Enrichment Analysis was performed using the software package provided through the Broad Institute (www.broadinstitute.org/gsea/index.jsp), on patient data obtained through the cBIO portal (www.cbioportal.org) and on cell-line data collected by the inventors after gene microarray (Affymetrix) analysis.

### Example 2: Role of PSMA in Calcium Homeostasis in Prostate Cancer

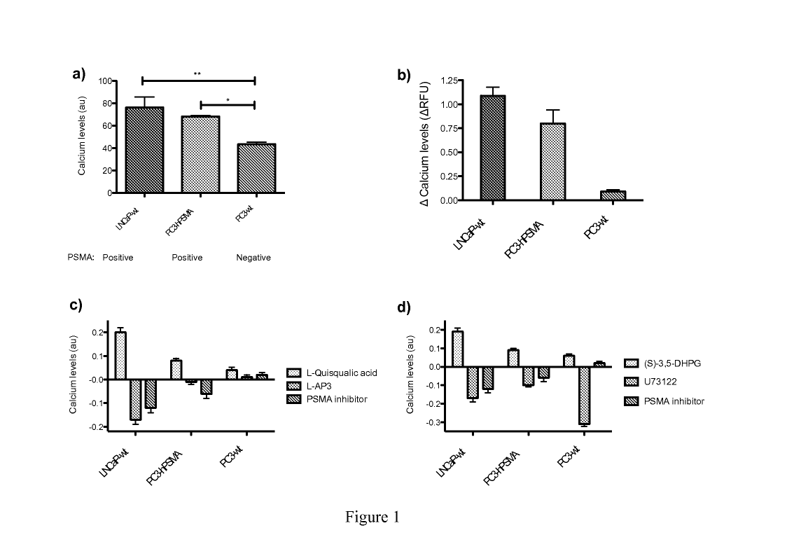

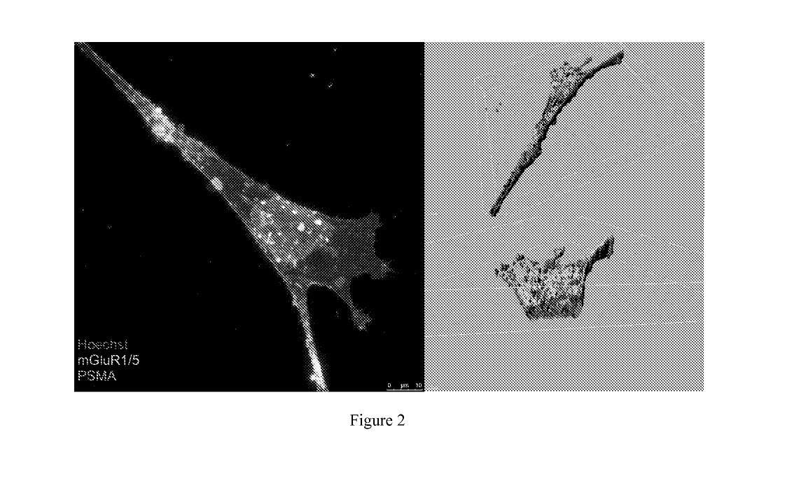

The effect of PSMA expression on calcium homeostasis in prostate cancer cell lines was evaluated using the fluorimetric Fluo4 assay kit (Invitrogen), according to the supplier's instructions. The plate was excited at 490 nm, and fluorescence emission was monitored at 520 nm with the SpectraMax M5 plate reader (Molecular Devices). Results indicated that the PSMA-expressing cells had higher cytoplasmic calcium concentrations. Expression of PSMA, such as by LNCaP-wt and PC3-hPSMA human prostate cancer cells, results in higher cytoplasmic calcium levels, when compared to cells lacking PSMA expression, such as PC3-wt (FIG. 1a). To examine calcium accumulation in the cytoplasm, the cells were treated with thapsigargin (1 μM, Tocris) immediately prior to taking measurements with the plate reader. The study revealed that the PSMA-expressing cells were more sensitive to thapsigargin than the PC3-wt cells, which led to significant buildup of calcium in their cytoplasm (FIG. 1b). Furthermore, we investigated whether PSMA facilitates calcium signaling via the mGluR Group I receptors through its enzymatic activity. The cells were treated with either L-Quisqualic acid (110 μM; mGluR I agonist, Tocris), L-AP3 (125 μM; mGluR I antagonist, Tocris) or a PSMA inhibitor (100 μM, Tocris), immediately prior to taking calcium measurements. In PSMA-expressing cells, we found that the calcium levels are regulated by PSMA's enzymatic activity and mGluR I, since inhibition of PSMA lowered calcium levels similar to inhibition of mGluR I (FIG. 1c). Additionally, we used (S)-3,5-DHPG (0.67 mM; mGluR I agonist, Tocris) and the Phospholipase C inhibitor U73122 (50 μM, Tocris), and found that PSMA inhibition resulted in reduction in calcium levels similar to inhibition of Phospholipase C, demonstrating that PSMA through its enzymatic function activates mGluR I, leading to Phospholipase C activation and initiation of calcium signaling (FIG. 1d).

### Example 3: PSMA Colocalizes with GluR

PSMA colocalizes with mGluR1/5 at the plasma membrane of prostate cancer cells. LNCaP cells were fixed with 4% paraformaldehyde and stained with the J591 PSMA antibody and a polyclonal antibody for mGluR1/5 (FIG. 2). LNCaP cells were seeded on 4-well chamber slides at a density of 1,000 cells per well. After overnight growth, the cells were fixed with 4% paraformaldehyde, followed by consecutive staining with the mouse J591 antibody to detect PSMA's extracellular motif, and a rabbit polyclonal antibody for type I mGluR (mGluR1/5 antibody, NB300-126, Novus Biologicals, Littleton, Colo.). The nucleus was stained with Hoechst 33342 (Life Technologies, Carlsbad, Calif.). The slides were imaged with a Leica upright confocal SP5 microscope.

### Example 4: PSMA's Enzymatic Activity Upregulates the Phosphorylation of Several Calcium-Dependent Signaling Effectors

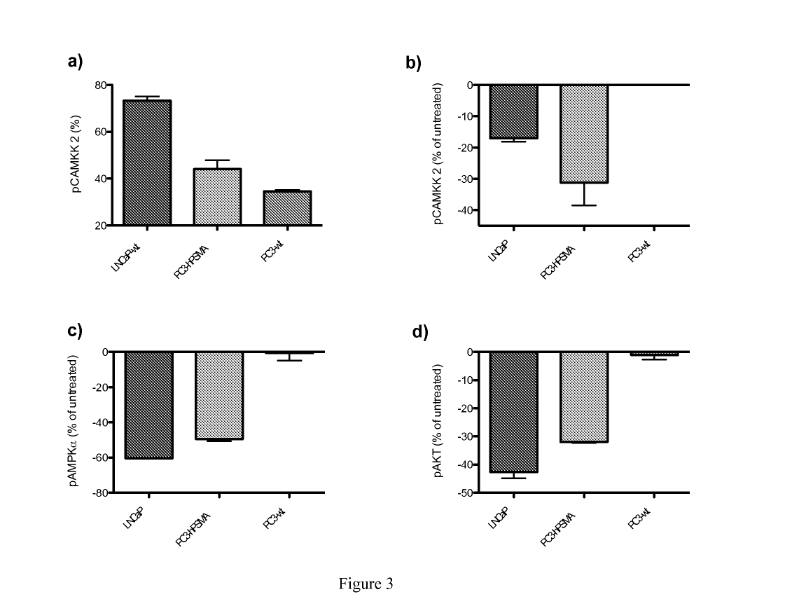

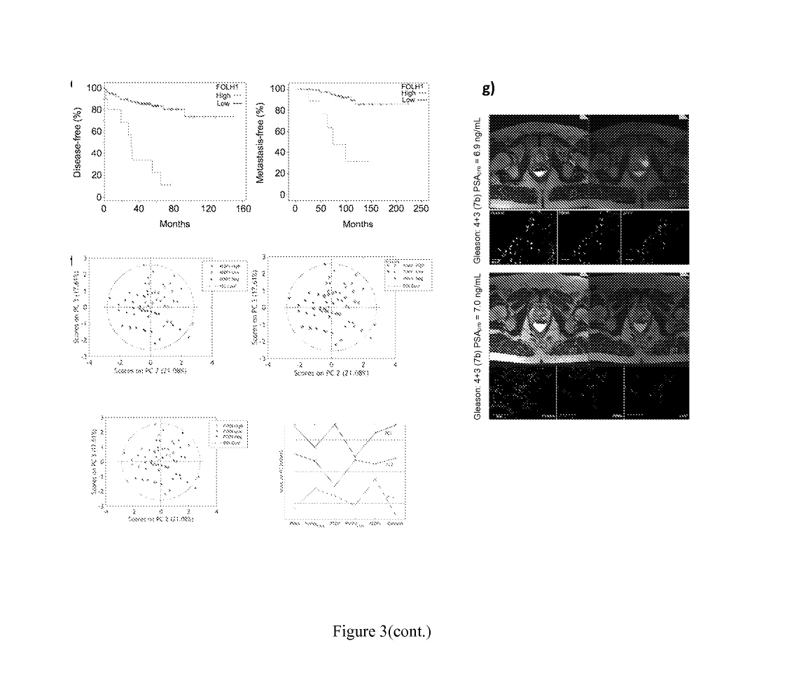

Phosphorylation state of calcium dependent downstream effectors was analyzed. Since activation of metabotropic glutamate receptors and their associated G proteins can lead to phosphorylation of downstream effectors, phosphorylation levels of key proteins relative to PSMA expression was examined. The NanoPro system from Protein Simple was used, which allowed the determination of global phosphorylation status based on the isoelectric point changes of the target protein. The samples were prepared according to the supplier's guidelines, following lysis of the cells with the NanoPro lysis buffer. All antibodies used were purchased from Cell Signaling, detecting the total protein levels of a target protein. The phosphorylation of the kinase CAMKK2 was higher in the PSMA-expressing cells (FIG. 3a) while in PSMA-negative cells (PC3-wt) the phosphorylation levels are substantially lower. Treatment with the PSMA inhibitor for 48 h (5 μM) decreased CAMKK2's phosphorylation in these cells with no effect in the cells lacking PSMA. (FIG. 3b). Likewise, the phosphorylation of AMPKα, a downstream target of CAMKK2, (FIG. 3c) and the key kinase AKT (FIG. 3d) was impaired in the PSMA-positive cells after treatment with the inhibitor, showing that PSMA through its enzymatic activity regulates major signaling pathways. It has been previously shown that either G protein-activated PI3K or calcium-activated mTORC2 phosphorylating PAK can cause Akt's phosphorylation. All graphs depict mean±s.e.m.

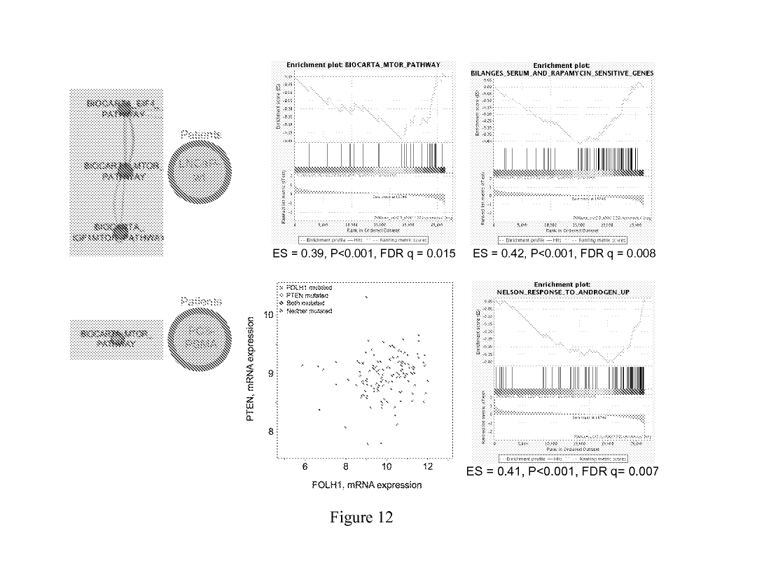

Data collected from human prostate cancer samples serves to confirm the role of PSMA in regulation of major signaling pathways. Expression level of PSMA (“FOLH1”) is demonstrated to be associated with faster biochemical occurrence and metastasis in prostate cancer patients (FIG. 3e). Additionally, principal component analysis of an 80-sample tissue microarray showed correlation between PSMA expression and phosphorylation of 4EBP1, which is downstream of AKT and mTOR (FIG. 30. As was found in the mouse model, patients that were PSMA positive through RFT had higher phosphorylated AKT than PSMA-negative individuals. Furthermore, in prostate cancer patients, expression of PSMA correlates with activation of the mTOR pathway, based on gene-set enrichment analysis (FIG. 12).

These data show that in prostate cancer PSMA's enzymatic activity regulates the phosphorylation of many critical signaling effectors, indicating its principal role in oncogenic signaling and suggesting unique therapeutic opportunities in prostate cancer.

### Example 5: PSMA Orchestrates a Complex Multicomponent Pro-Oncogenic Repertoire

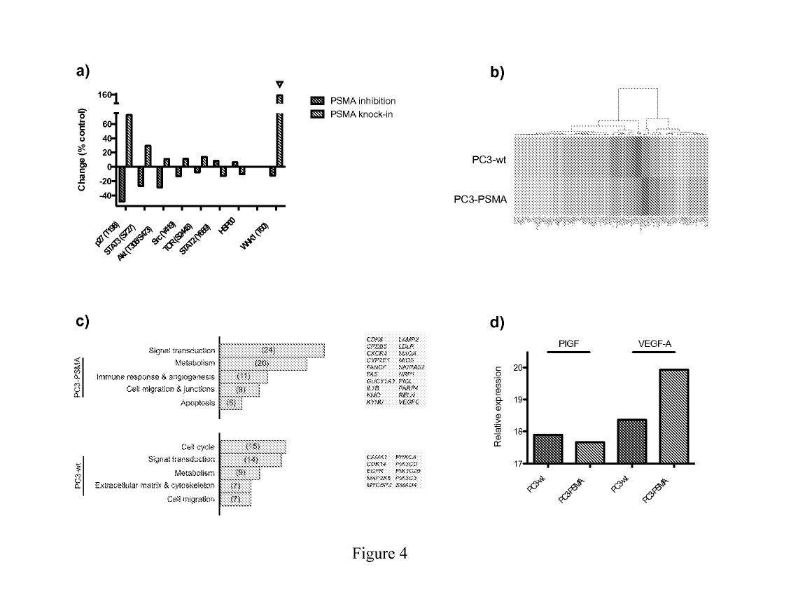

Considering that PSMA inhibition affected the phosphorylation of some key kinases, a human kinome-profiling array from R&D Systems was utilized to obtain a wider view of other kinases being regulated by this protein. LNCaP-wt cells were grown in the presence of a PSMA inhibitor (48 h, 5 μM) and PC3-PSMA cells were grown under puromycin-induced selection. Control cells included LNCaP-wt and PC3-wt cells grown for 48 h in complete RPMI media. The cells were lysed and processed according to the array's protocol, and the array was performed according to its supplier's guideline. Imaging of the array's membranes was performed after film exposure in the presence of a chemiluniescent substrate and HRP-conjugated detecting antibodies, processed in a dark room. Results showed that inhibition of PSMA affected the phosphorylation of many proteins including TOR, p27, and Src, whereas PSMA expression in PC3 cells increased the phosphorylation of these proteins (FIG. 4a). The phosphorylation of many important effectors decreased upon treatment of LNCaP-wt cells with the PSMA inhibitor, while expression of PSMA by PC3 cells (PC3-hPSMA) reciprocally upregulated the phosphorylation of these proteins.

Genomic analyses using the Affymetrix U133A 2.0 gene array showed that multiple genes were upregulated in PC3 cells expressing PSMA (FIG. 4b). Classification of these genes based on their function using the online DAVID functional annotation platform revealed that many of the genes upregulated in PSMA-expressing PC3 cells were involved in signal transduction, metabolism, and angiogenesis. PSMA-null cells have higher mRNA levels of genes involved in cell cycle regulation and signaling (FIG. 4c). Among the most striking findings was the upregulation of the expression of VEGF-A in PSMA-expressing cells without affecting the levels of PIGF. Expression of PSMA in PC3 cells (PC3-PSMA) upregulated the levels of the pro-metastatic/angiogenic effector VEGF-A, without affecting the levels of PIGF This demonstrates PSMA's role in prostate cancer angiogenesis and general tumor neovascularization (FIG. 4d). These data demonstrate the pleiotropic effect that PSMA exerts on PCa development and advancement, suggesting that early therapeutic interventions that effectively inhibit PSMA might have great clinical potential and increase survival.

### Example 6: PSMA Contributes to Prostate Cancer's Advancement

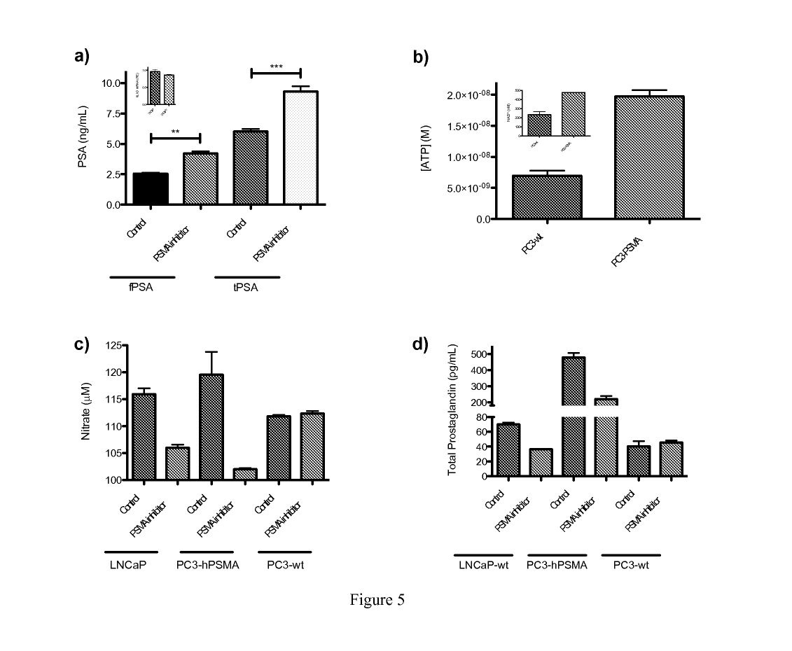

Since PSMA regulates Akt phosphorylation, it was investigated whether inhibition of PSMA and subsequent downregulation of Akt activity affected the status of the androgen receptor (AR) pathway. LNCaP-wt cells, which have functional androgen receptor (AR), were grown for 48 h in the presence of PMSA inhibitor (5 μM), and the cells' culturing medium was screened for PSA (DELFIA PSA, Perkin Elmer), since PSA levels are regulated by the AR pathway. Results showed that inhibition of PSMA increased PSA levels, due to overactivation of the AR signaling cascade via relief of the Akt-mediated negative feedback (FIG. 5a). Inhibition of PSMA did not affect PSA's mRNA levels.

Since Akt is also involved in cellular metabolism, it was determined whether expression of PSMA in PC3 cells alters their metabolic pathways, by measuring the levels of NAD+ and ATP. For NAD+ measurements, a spectrophotometric assay was purchased from Cayman Chemicals, whereas ATP quantification was performed with the StayBrite ATP Assay from Biovision. All assays were performed according to the corresponding supplier's protocol. PC3-PSMA cells had higher levels of both NAD+ and ATP than the PSMA-negative cells (PC3-wt), showing that PSMA-expression alters prostate cancer bioenergetics and increases ATP generation by shifting energy production from aerobic glycolysis to oxidative phosphorylation (FIG. 5b).

Furthermore, it was examined whether PSMA inhibition affects the activity of nitric oxide synthase and the levels of prostaglandins, since both Akt and Ca+2 regulate these processes. The activity of nitric oxide synthase was assessed by quantifying the sample's total nitrate levels with a kit from Cayman Chemicals, whereas the levels of total prostaglandins secreted in the cell's medium were measured with the total prostaglandin kit from Cayman Chemicals. Inhibition of PSMA was performed with a PSMA inhibitor (5 μM) at 48 h at 37° C., 5% CO₂. All samples were prepared according to the kits' instructions, with the results showing that inhibition of PSMA decreased nitrate and prostaglandin levels. Inhibition of PSMA decreased nitric oxide synthase activity, reflected in lower nitrate levels. Treatment with the inhibitor did not affect nitrate concentration in PC3-wt cells that do not express PSMA. The total levels of secreted prostaglandins decreased after treatment of LNCaP and PC3-PSMA cells with the PSMA inhibitor, as opposed to cells lacking PSMA (PC3-wt). (FIG. 5c-d). These data show that PSMA negatively regulates the AR pathway and switches prostate cancer's metabolism to oxidative phosphorylation, which is encountered in advanced and metastatic lesions. PSMA also regulates the levels of potent angiogenic, inflammatory, and metastatic effectors, which contribute in prostate cancer's advancement and undermine effective treatment.

### Example 7: PSMA Affects Angiogenesis and Tumor Oxygenation In Vivo

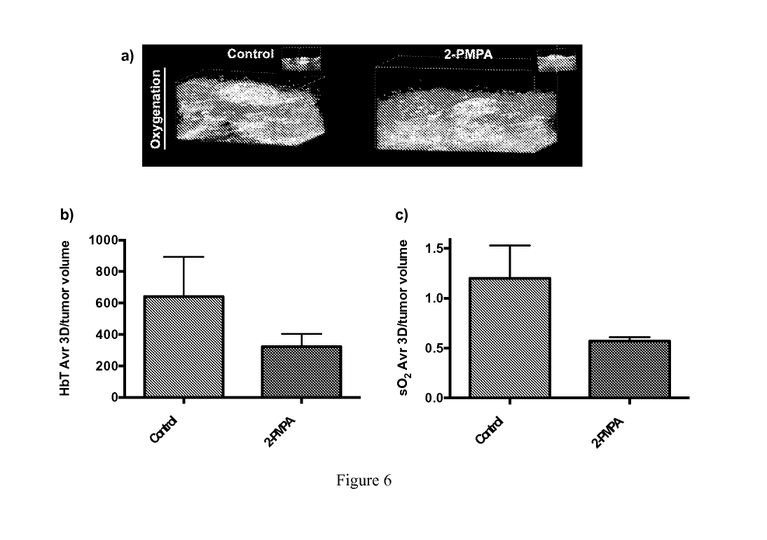

In vivo studies with male athymic, nude mice that were implanted with LNCaP-wt xenografts on their flanks and treated daily with saline or PSMA inhibitor (2-PMPA, 0.4 mM, 100 μL retro-orbital injection) upon xenograft implantation showed that inhibition of PSMA caused lower tumor vascularization and lower tumor oxygenation, which was assessed using VisualSonics' photoacoustic system based on the different light absorbing properties of oxy- and deoxy-hemoglobin. The tumors used in the study had roughly the same size. The animals treated with the inhibitor had tumor with lower total hemoglobin levels, indicating that PSMA plays a role in tumor vascularization. (FIG. 6 a-b). In addition to tumor vascularization, inhibition of PSMA affected the tumor's metabolic programming, which resulted lower consumption of oxygen. (FIG. 6c)

### Example 8: Inhibition of PSMA's Enzymatic Activity Improves the Cytotoxicity of Many Chemotherapeutics

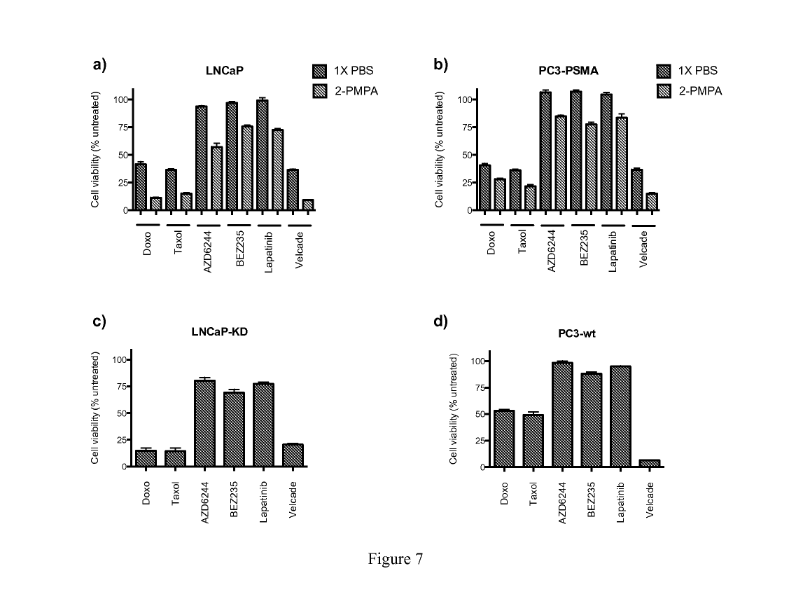

Given that PSMA affects the phosphorylation status of multiple targets, it was investigated whether inhibition of PSMA can counteract resistance to various chemotherapeutics used in the clinic or under clinical trials. LNCaP and PCS3-PSMA cells were seeded at a density of 2,500 cells per well in a 96-well format, and after 48 hours growth the cells were treated with the drugs (Doxorubicin (Adriamycin; DNA intercalator), Taxol (Paclitaxel; microtubule stabilizer), AZD6244 (Selumetinib; MEK1 & ERK1/2 inhibitor), BEZ235 (Dactolisib; PI3K & mTOR inhibitor), Lapatinib (EGFR and ErbB2 inhibitor), or Velcade (Bortezomib; 20S proteasome inhibitor), 200 nM final concentration in 1×PBS) or with the drugs (200 nM final concentration) and 2-PMPA (5 μM final concentration). After overnight incubation, cell viability was assessed fluorimetrically with the Alamar blue method (Invitrogen), according to the supplier's protocol. Inhibition of PSMA enhanced the toxicity of all chemotherapeutics in PSMA-expressing cells, whereas the LNCaP cells that lacked PSMA (LNCaP-KD) were more sensitive to all drugs than their parental cell line (LNCaP). PC3-wt cells had similar cytotoxic profiles to the PSMA-expressing PC3 cells (PC3-PSMA) for all drugs, other than Velcade. This might be attributed to the different oxidative stress burden between these two cell lines, making PC3-wt cells more sensitive to Velcade. (FIG. 7a-d). The PSMA inhibitor alone caused no cytotoxic effect for the study's time-course. These data confirm that PSMA provides chemoresistance and survival advantage to prostate cancer.

### Example 9: Expression of PSMA Provides Resistance to Chemotherapy that Increases the Intracellular Levels of Reactive Oxygen Species

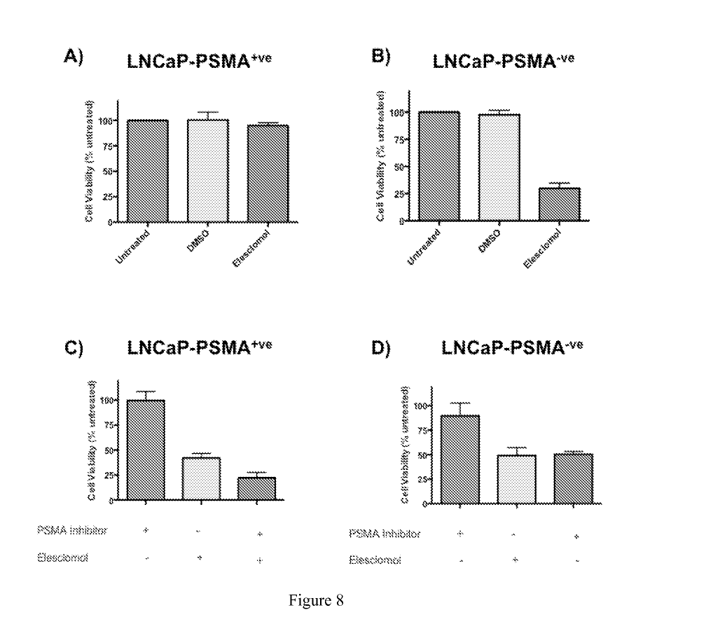

Cell viability of (A) PSMA-expressing (LNCaP-PSMA+ve) and (B) PSMA-negative cells (LNCaP-PSMA-ve where PSMA expression was knocked down with shRNA) was determined via the fluorescence-based Alamar Blue method (λex=565 nm and λem=585 nm). Cell viability was assessed 48 h after drug administration with either vehicle (DMSO) or the ROS-generating drug Elesclomol (250 nM, Selleck Chemicals). Inhibition of PSMA's enzymatic activity (C and D) abrogates drug resistance and renders the cells susceptible to cell death. Cell viability of (C) PSMA-expressing and (D) PSMA-negative cells treated with a selective PSMA inhibitor (2-PMPA, 250 nM). The cells were treated for 48 h, and cell viability was determined with the Alamar Blue method. (Mean±SD, n=4) (FIG. 8).

### Example 10: Inhibition of PSMA In Vivo Hampers Tumor Growth and Improves Survival

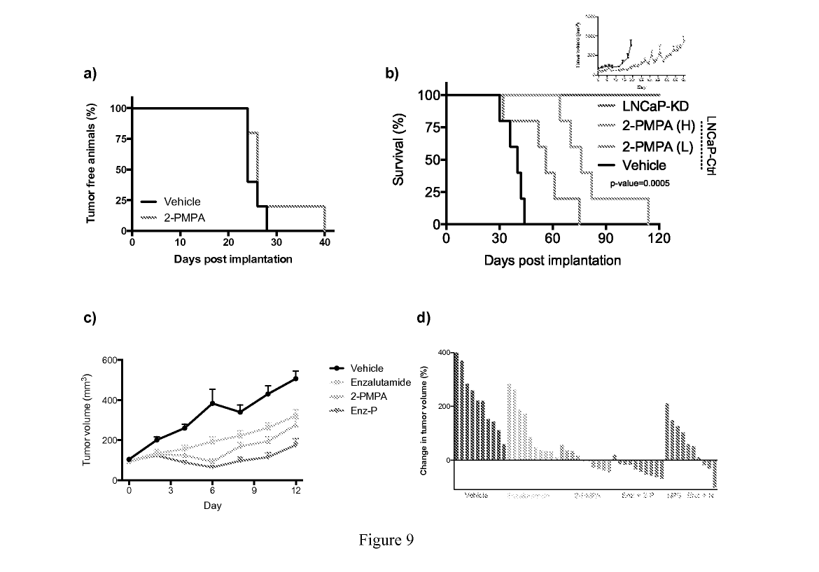

Tumor growth was examined using daily treatment of male athymic, nude mice with LNCaP xenografts on their flanks Each animal's flanks were injected with 3,000,000 cells in 100 μL matrigel, and the animals were immediately treated with 2-PMPA (0.4 mM, 100 μL retro-orbital injection). Tumor volume was assessed by measuring the tumor with microcalipers. Results showed that inhibition of PSMA did not affect tumor initiation (FIG. 9a), but it resulted in smaller tumors that grew slowly and extended the animals survival (FIG. 9b). In a different study, male athymic, nude mice with LNCaP xenografts were treated daily post tumor detection, with either enzalutamide (0.15 mM, 100 μL retro-orbital injection), 2-PMPA (3 mM, 100 μL retro-orbital injection) or both compounds (0.15 mM enzalutamide and 3 mM 2-PMPA, 100 μL retro-orbital injection). Combination treatment with the PSMA inhibitor (2-PMPA) and the AR antagonist (enzalutamide) resulted in slower tumor growth and early tumor regression, suggesting that PSMA's inhibition might provide an important therapeutic intervention window during the early stages of the disease. (FIG. 9c-d).

### Example 11: PSMA Levels Correlate to Poor Chemotherapy Response In Vivo

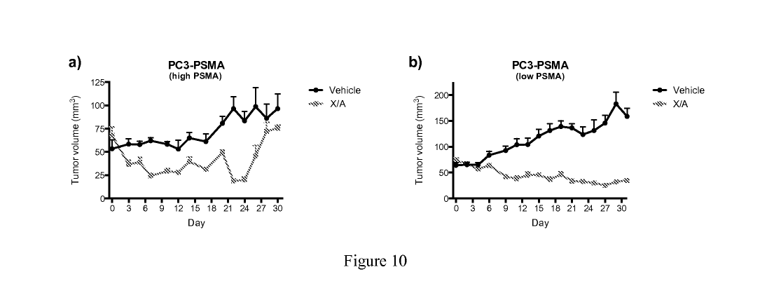

As PSMA through Akt upregulates mTOR and angiogenic signaling tumor therapy response through inhibition of the mTOR and angiogenic pathways was investigated. PC3-PSMA xenografts were implanted on male athymic, nude mice (3,000,000 cells per flank in matrigel, 100 μL subcutaneous), using cells that had high or low PSMA levels. After all mice developed tumors on their flanks, the treatment course was initiated, where every other day the mice were treated with either vehicle (DMSO) or a combination of AZD8055 and XL184 (AZD8055 0.4 mM and XL184 8 μM 100 μL retro-orbital injection; tumor dimensions measured with calipers). At the end of the study, the mice that were treated with the drugs and had lower PSMA levels showed tumor regression, as opposed to the counterparts that had xenografts with higher PSMA levels (FIG. 10a-b).

### Example 12: Identification of Genes Whose Gene Expression is Upregulated Due to PSMA Expression and Activity

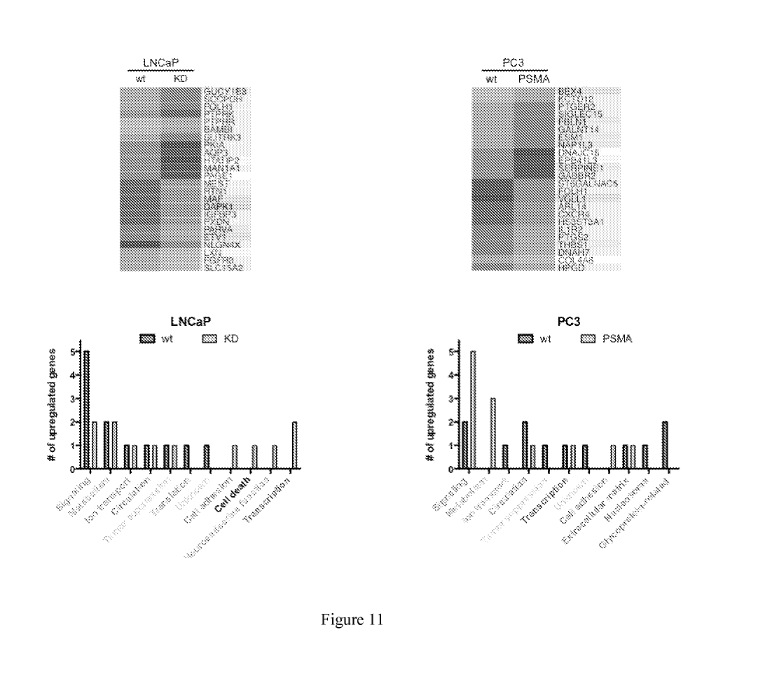

Classification of genes into families of cellular processes also shows that PSMA expression upregulates the expression of signaling effectors in prostate cancer. LNCaP wt: PSMA-positive, LNCaP KD: PSMA-negative, PC3 wt: PSMA-negative, PC3-PSMA: PSMA-positive. (FIG. 11).

### Example 13: PSMA's Role in Prostate Cancer

These findings demonstrate that through its enzymatic activity and ability to process (poly)glutamated substrates, including NAAG and folates, PSMA activates metabotropic Glutamate Receptors Group I, which initiate a downstream signaling cascade that increases cytosolic calcium levels. The released calcium further activates various signaling effectors, alters metabolism and primes the tumor and its environment for metastasis (FIG. 13).

### Example 14: Modified Amplex Red Glutamic Acid Assay for Quantification of PSMA Levels in Clinical Samples

The modified PSMA Amplex Red Glutamic Acid/Glutamate Oxidase Assay kit (Life Technologies, Carlsbad, Calif.) utilizes folic acid instead of glutamic acid, since folic acid consists of a pteroyl moiety linked to glutamic acid via an amide bond. In the presence of PSMA, the amide bond is cleaved liberating the pteroyl group and glutamate, where the glutamate can be oxidized by the Amplex Red assay's glutamate oxidase to produce α-ketoglutarate, ammonia, and hydrogen peroxide. For signal enhancement, a transamination reaction occurs, where L-glutamate transaminase converts α-ketoglutarate to glutamate. Once collected, EPS urine samples can be used immediately or stored at −80° C. For PSMA quantification, these samples were pre-incubated for 24 h with the folic acid substrate (2 mM) at room temperature, in a 50 mM HEPES and 0.1 M NaCl buffer. Adjustment of total protein concentration was performed through dilutions with this buffer. The assay used positive controls, which consisted of recombinant PSMA (4 nM, 10 nM, 20 nM, 40 nM, and 100 nM, R&D Systems, Minneapolis, Minn.). The positive controls were supplemented with 2 mM of the folic acid substrate, in order to allow signal normalization and subsequent PSMA quantification. At the end of the 24 h incubation, the Amplex Red Glutamic Acid assay was performed, where the samples were supplemented with 100 μM Amplex Red reagent containing 0.25 U/mL horseradish peroxidase, 0.08 U/mL L-glutamate oxidase, 0.5 U/mL L-glutamate-pyruvate transaminase, and 200 μM L-alanine in 1× reaction buffer (Life Technologies, Carlsbad, Calif.). Results were obtained with a microplate reader that could detect fluorescence (SpectraMax M5, Molecular Devices, Sunnyvale, Calif.), as well as with a small animal imaging system (IVIS200, Perkin Elmer, Waltham, Mass.).

### Example 15: Activatable Agent for Quantification of PSMA Levels in Clinical Samples

The PSMA activatable agent consists of a glutamate substrate conjugated to luciferin via an amide bond. In the presence of PSMA, the amide bond is cleaved liberating glutamate and luciferin, which can be detected by luciferase. Once collected, EPS urine samples can be used immediately or stored at −80° C. For PSMA quantification, these samples were pre-incubated for 24 h with the glutamate-luciferin substrate (2 mM) at room temperature, in a 50 mM HEPES and 0.1 M NaCl buffer. Adjustment of total protein concentration was performed through dilutions with this buffer. The assay used positive controls, which consisted of recombinant PSMA (4 nM, 10 nM, 20 nM, 40 nM, and 100 nM, R&D Systems, Minneapolis, Minn.). The positive controls were supplemented with 2 mM of the glutamate-luciferin substrate, in order to allow signal normalization and subsequent PSMA quantification. At the end of the 24 h incubation, the luciferase enzyme assay was performed, where the samples were supplemented with 2 nM of Firefly Luciferase (Roche, San Francisco, Calif.) in bioluminescence buffer [40 mM Tris-acetate, 1 mM EDTA, 1 mM DTT, 3.45 mM ATP, 0.2 M NaCl, 5.7 mM MgSO4, and 0.76 mM coenzyme A (pH 7.6)]. Results were obtained with a microplate reader that could detect luminescence (SpectraMax M5, Molecular Devices, Sunnyvale, Calif.), as well as with a small animal imaging system (IVIS200, Perkin Elmer, Waltham, Mass.).

We claim:

1. A method of treating or preventing cancer comprising:
administering a therapeutically effective amount of a chemotherapeutic to a patient who is sensitized to the chemotherapeutic in that the patient has received a PSMA inhibitor.

2. In a method of treating or preventing cancer by administering therapy with a chemotherapeutic agent, the improvement that comprises:
administering a PSMA inhibitor to the patient prior to, concomitant with, or after initiation of the therapy.

3. The method of claim 1, comprising the step of administering to the patient a therapeutically effective amount of a PSMA inhibitor prior to administration of the chemotherapeutic.

4. The method of any one of the preceding claims, comprising the step of administering to the patient a therapeutically effective amount of a PSMA inhibitor concurrent with administration of the chemotherapeutic.

5. A method of treating or preventing cancer comprising administering to a subject suffering from or susceptible to a refractory cancer a therapeutically effective amount of a PSMA inhibitor.

6. The method of any one of the preceding claims, wherein the cancer is refractory to treatment with an androgen receptor inhibitor or hormone deprivation.

7. The method of any one of the preceding claims, wherein the cancer is refractory to treatment with a chemotherapeutic.

8. A method of treating or preventing cancer comprising steps of:
1) identifying a patient suffering from or susceptible to a cancer characterized by high levels of PSMA; and
2) administering a therapeutically effective amount of a PSMA inhibitor.

9. The method of claim 8, wherein a high level of PSMA is indicated when the concentration of PSMA in the patient's test tissue sample from is higher than the concentration of PSMA from the patient's healthy tissue sample.

10. The method of claim 8, wherein a high level of PSMA is indicated when the concentration of PSMA in the patient's test tissue sample from is higher than the normal concentration of PSMA in the patient population.

11. The method of claim 8, further comprising the step of administering a therapeutically effective amount of a chemotherapeutic concurrent with or subsequent to administration of the PSMA inhibitor.

12. A method for reducing resistance to a chemotherapeutic in a patient comprising administering a therapeutically effective amount of a PSMA inhibitor concurrent with or prior to administration of the chemotherapeutic.

13. A method for sensitizing tumor cells to a chemotherapeutic comprising treating the tumor cells with a PSMA inhibitor.

14. The method of any one of the preceding claims, wherein chemotherapeutic is selected from the group consisting of topoisomerase I inhibitors, topoisomerase II inhibitors, microtubule active compounds, compounds which induce cell differentiation processes, compounds targeting/decreasing a protein or lipid kinase activity and further anti-angiogenic compounds, compounds which target, decrease, or inhibit the activity of a protein or lipid phosphatase, anti-androgens, proteasome inhibitors, and MEK inhibitors.

15. The method of claim 13 or 14, wherein the chemotherapeutic is selected from doxorubicin, taxol, AZD6244, BEZ235, lapatinib, velcade, and enzalutamide.

16. A method of inhibiting cancer cell migration comprising administering to a patient suffering from or susceptible to cancer a therapeutically effective amount of a PSMA inhibitor.

17. A method of inhibiting neovascularization comprising administering to a patient suffering from or susceptible to cancer a therapeutically effective amount of a PSMA inhibitor.

18. The method of claim 17, wherein the neovascularization is tumor neovascularization.

19. The method of any one of the preceding claims, wherein the cancer is selected from the group consisting of squamous cell cancer (e.g. epithelial squamous cell cancer), lung cancer including small-cell lung cancer, non-small cell lung cancer, adenocarcinoma of the lung and squamous carcinoma of the lung, bone cancer, cancer of the peritoneum, esophageal cancer, eye cancer, skin cancer, gastric or stomach cancer including gastrointestinal cancer, pancreatic cancer, glioblastoma, cervical cancer, ovarian cancer, liver cancer, bladder cancer, gallbladder cancer, hepatoma, laryngeal cancer, oral cancer, brain cancer, breast cancer, colon cancer, rectal cancer, colorectal cancer, endometrial or uterine cancer, salivary gland carcinoma, kidney or renal cancer, neuroendocrine cancer, prostate cancer, vaginal cancer, vulval cancer, testicular cancer, thyroid cancer, urethral cancer, hepatic carcinoma, anal carcinoma, penile carcinoma, and head and neck cancer.

20. The method of claim 19, wherein the cancer is of the breast, lung, or colon.

21. The method of any one of the preceding claims, wherein the cancer comprises a solid tumor.

22. The method of claim 21, wherein the solid tumor is other than a prostate or sarcoma tumor.

23. The method of any one of the preceding claims, wherein the PSMA inhibitor is used at a concentration that alone causes no cytotoxic effect.

24. The method of any one of the preceding claims, wherein the PSMA inhibitor is used at a concentration that alone slows but does not reverse tumor growth.

25. The method of any one of the preceding claims, wherein the therapeutically effective amount of PSMA inhibitor is an amount effective to inhibit or decrease metastatic spread of cancer.

26. The method of any one of the preceding claims, wherein PSMA inhibitor is selected from the group consisting of (RS)-2-PMPA, (R)-2-PMPA, (S)-2-PMPA, (RS)-GPI5232, (S)-GPI5232, RS)-2-MMPA, (R)-2-MMPA, (S)-2-MMPA, PBDA, (R,R)/(S,S)-PBDA, (S,S)/(R,R)-PBDA, meso-PBDA, (S)-Glu-C(O)-(S)-Glu, (R)-Glu-C(O)-(R)-Glu, (R)-Glu-C(O)-(S)-Glu, [¹¹C]DCMC, [¹²⁵I]DCIT, VA-033, ZJ43, ZJ11, ZJ17, ZJ38, CTT54, TG97, DBCO-PEG₄-AH₂-TG97, DBCO-PEG(4)-CTT-54, DBCO-PEG(4)-CTT-54.2, pemetrexed, methotrexate, a pseudoirreversible inhibitor peptidomimetic, a steroid-derived phosphoramidate inhibitor, an alphabody, a DARPin, and combinations thereof.

27. The method of claim 26, wherein the PSMA inhibitor is 2-PMPA.

28. A method of treating cancer in a patient suffering from or susceptible to the cancer, comprising steps of:
i) determining the amount of PSMA present on a patient's tumor; and
ii) administering a suitable chemotherapeutic to the patient;
wherein a high level of PSMA indicates the patient should be treated with an elevated level of chemotherapy.

29. A method of treating cancer in a patient suffering from or susceptible to cancer, the method comprising steps of:
administering to a patient who:
a) is receiving therapy with a chemotherapeutic agent; and
b) shows a high level of PSMA;
an elevated dose of the chemotherapeutic agent.

30. The method of claim 28 or 29, wherein an elevated level of chemotherapy comprises increasing the concentration of one or more chemotherapeutics the patient is administered.

31. The method of any one of claims 28-30, wherein an elevated level of chemotherapy comprises administering one or more additional chemotherapeutics to the patient.

32. The method of claim 8, 28, 29, 30, or 31, wherein a high level of PSMA is indicated when a patient has a PSMA level above about 1-5 pg/mL, about 5 pg/mL, about 10 pg/mL, about 20 pg/mL, about 30 pg/mL, about 50 pg/mL, about 75 pg/mL, about 100 pg/mL, about 150 pg/mL, about 200 pg/mL, about 250 pg/mL, about 500 pg/mL, or about 1000 pg/mL.

33. The method of claim 8, 28, 29, 30, or 31, wherein a high level of PSMA is indicated when a sample of expressed prostatic secretion incubated with a detectable folic acid substrate shows a fluorescence radiance of greater than 50, 70, 80, 90, 100, 125, 150, or 200, normalized to volume.

34. The method of claim 28 or 29, further comprising administering to the patient a therapeutically effective amount of a PSMA inhibitor.

35. The method of any one of the preceding claims, further comprising administering to the patient an androgen receptor inhibitor, a mTOR inhibitor, or a combination thereof.
